(19) United States
(12) Patent Application Publication
(10) Pub. No.: US 2010/0199984 A1
(43) Pub. Date: Aug. 12, 2010

(54) BREATH ACTUATED NASAL PUMP

(75) Inventors: Robert C. Williams, III, Raleigh, NC (US); Daniel M. Deaton, Apex, NC (US); Perry Genova, Chapel Hill, NC (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/598,870

(22) PCT Filed: Apr. 1, 2008
(86) PCT No.: PCT/US2008/059056

§ 371 Date: Apr. 23, 2010

Related U.S. Application Data
(60) Provisional application No. 60/909,569, filed on Apr. 2, 2007.

(51) Int. Cl. A61M 11/00

(52) U.S. Cl. 128/200.23

(57) ABSTRACT

A breath actuated nasal drug delivery device includes a housing having a spray port; a reservoir housing a liquidous medicament; and a selectively actuable pump. A breath actuated triggering mechanism includes a mouthpiece and a diaphragm. A pressure differential across the diaphragm causes an elastically deformable drive element (e.g., spring) to release stored potential energy, causing the pump's plunger to move to thereby pump the medicament to the spray port. A cocking lever can be pivotally mounted to the housing for causing the drive element to store potential energy. The triggering mechanism can include a cam element pivotally attached to the housing and the diaphragm that rotates in response to movement of the diaphragm. A follower element can be pivotally connected to the plunger for movement therewith; the follower element releasably engages the cam element. Related methods are also described.

### FIELD OF THE INVENTION

The present invention relates to a nasal drug delivery device for delivery of liquid medicament to the nasal cavity, particularly the nasal epithelia.

### BACKGROUND OF THE INVENTION

Nasal delivery of pharmaceutical products can be useful both for treating diseases or disorders in the nasal passages themselves and for treating systemic and/or neurological disorders. However, it has been observed that particle or droplet size has significant impact on absorption when administering drugs via the nose and the nasal epithelia. Smaller droplets have been shown to impact on the higher nasal turbinates which promotes better absorption into the body. On the other hand, droplets that are too small, and/or are delivered at too high a velocity, can be carried beyond the nasal passageway and undesirably find their way into the pulmonary region. Indeed, FDA Guidelines require testing to demonstrate that only a minimal amount of drug from a nasal delivery device be deposited beyond the nasal passageway and find its way into the pulmonary region.

Delivery of pharmaceutical products via the nasal epithelia offers a drug delivery route that can bypass the “blood brain barrier” in some situations. As such, the nasal epithelia can be a useful route both for delivering pharmaceutical products for treating diseases or disorders in the nasal passages themselves and for delivering pharmaceutical products for treating systemic and/or neurological disorders. However, it has been observed that particle or droplet size has significant impact on absorption when administering drugs via the nose and the nasal epithelia. Smaller droplets have been shown to impact on the higher nasal turbinates which promotes better absorption into the body. On the other hand, droplets that are too small, and/or are delivered at too high a velocity, can be carried beyond the nasal passageway and undesirably find their way into the pulmonary region. Indeed, FDA Guidelines require testing to demonstrate that only a minimal amount of drug from a nasal delivery device be deposited beyond the nasal passageway and find its way into the pulmonary region.

Traditional devices for supplying drugs to the nasal epithelia include syringed nose drops, pump spray devices, and fluorinated propellant metered dose inhalers (MDI). These traditional devices have not generally been able to achieve the particle sizes necessary to maximize efficacy while helping mitigate undesired pulmonary absorption. For example, both eye dropper type devices and simple spray devices typically present medicament into the nasal cavity in a stream. The result is that much of the medicament simply runs out of the patient's nose, and only a small amount of the drug is absorbed, with even less of the drug reaching the nasal epithelia.

Newer pump type devices have increased ability to reduce the particle size of the medicament but have drawbacks of their own. Most pump devices rely on the user's hand strength to overcome a spring pressure in the pump, and create a pumping action. However, many individuals end up with less than optimal sprays produced from such pumps because of the variation in action of applying the necessary power to the pump and/or the variability in hand strength. Other devices, known as metered dose propellant type devices, tend to produce good particle size, but at an undesirably high effective velocity. The pressure of the propellant in these devices tends to cause the drug to escape the nasal passageways and thus be deposited in the lungs or other portions of the pulmonary region.

Further, it has been observed that when a human exhales air from the lungs though the mouth, and particularly in instances where the exhalation is impeded by something that creates a backpressure in the pulmonary system, the soft palate operates to isolate and insulate the nasal pharynx from the remainder of the pulmonary system. That is, the soft palate acts as a natural check valve preventing the flow of air between the lungs and the nasal cavity. Thus, it is believed that nasal drug delivery can be improved if the patient is exhaling orally while the drug is being sprayed into the nasal passages. One nasal delivery system that takes advantage of this is shown in U.S. Patent Application Publication 2006/0289007, which is incorporated herein by reference. The '9007 publication discloses using a pressurized canister of the type typically found in metered dose inhalers, which may not be appropriate for all situations.

Accordingly, there remains a need for alternative means of delivering a desired amount of drug to the nasal epithelia, advantageously in desired particle size distribution, and/or at a desired velocity.

### SUMMARY OF THE INVENTION

Various embodiments of the present invention are intended to provide a nasal drug delivery device and/or a related method of delivering drugs nasally, typically using a breath actuation triggering approach.

In one embodiment, a nasal drug delivery device includes a housing having a spray port; a reservoir housing a liquidous medicament; and a selectively actuable pump supported by the housing and operatively connecting the reservoir to the spray port. A drive element (e.g., a spring) is operatively connected to the plunger and is elastically deformable between a higher potential energy state and a lower potential energy state. A breath actuated triggering mechanism associated with the housing includes a mouthpiece and a diaphragm, and controls the release of the drive element from the higher potential energy state to the lower potential energy state. A pressure differential across the diaphragm, higher towards the mouthpiece, causes the drive element to change from the higher state to the lower state, and the pump's plunger moves in response thereto to thereby cause the pump to supply the medicament to the spray port. A cocking lever can be pivotally mounted to the housing for movement between a first position and a second position, with movement of the cocking lever to the second position causing the drive element to elastically store potential energy. A second elastic element can act on the plunger in opposition to the firing element to provide a reset bias to the plunger. The triggering mechanism can further include a cam element pivotally attached to the housing and the diaphragm, wherein the cam element rotates in response to movement of the diaphragm. A follower element can be pivotally connected to the plunger for movement therewith and the follower element releasably engages the cam element. When the follower element is released from engagement with the cam element, the drive element is released to power the pump.

In another embodiment, a nasal drug delivery device includes a housing having a spray port; a reservoir housing a liquidous medicament; a selectively actuable pump supported by the housing and operatively connecting the reservoir to the spray port. An elastically deformable drive element is operatively connected to the plunger. A breath actuated triggering mechanism associated with the housing includes a mouthpiece and a diaphragm. The device is moveable between a cocked configuration and a delivery configuration. In the cocked configuration, the drive element is held in a first relatively higher potential energy state and the pump's plunger is relatively undepressed; in the delivery configuration, the drive element is in a relatively less potential energy state and the pump's plunger is relatively depressed. Blowing into the mouthpiece causes the diaphragm to move so as to release the drive element from the first state and thereby depress the plunger.

In another embodiment, a nasal drug delivery device includes a housing having a spray port; a reservoir housing a liquidous medicament; and a selectively actuable pump supported by the housing and operatively connecting the reservoir to the spray port. A cocking lever is pivotally mounted to the housing for movement between a first position and a second position. A first elastic element is operatively disposed between the plunger and the cocking lever, and movement of the cocking lever to the second position causes the elastic element to store energy. A trigger mechanism includes a diaphragm connected to the pump's plunger via a selectively breakable linkage. Inward movement of the diaphragm causes the linkage to break, thereby causing the pump's plunger to be depressed by the release of the energy stored in the elastic element.

In another embodiment, a method of administering a medicament nasally to a user includes providing a nasal delivery device, the nasal delivery device including: a housing having a spray port; a reservoir housing a liquidous medicament; a selectively actuable pump supported by the housing and operatively connecting the reservoir to the spray port; a cocking lever pivotally mounted to the housing for movement between a first position and a second position; and a first elastic element disposed operatively between the pump's plunger and the cocking lever. The method further includes storing energy in the first elastic element by moving the cocking lever to the second position while resisting movement of the plunger; and blowing into a mouthpiece associated with the housing, and, in response thereto, releasing the stored energy to depress the plunger to thereby cause delivery of a portion of the medicament into the nasal passages of a user. The releasing of stored energy can include deforming a diaphragm in response to blowing into the mouthpiece, and can further include breaking a selectively breakable linkage connecting the diaphragm to the plunger in response to inward deformation of the diaphragm.

In another embodiment, a method of administering a medicament nasally to a user includes providing a nasal delivery device including: a housing including a distal end portion and a proximal end portion and including a spray port disposed proximate the proximal end portion; the spray port configured to be inserted in a human user's nose; a reservoir housing a liquidous medicament; a manually powered pump supported by the housing and operatively connecting the reservoir to the spray port; an elastically deformable drive element operatively connected to the plunger; a breath actuated triggering mechanism associated with the housing and including a mouthpiece and a diaphragm; and the triggering mechanism controlling release of the drive element from a higher potential energy state to a lower potential energy state. The method further includes: disposing the proximal end portion proximate the user's face and the distal end portion distal from the user's face; a forward direction defined as extending from the distal end portion toward the proximal end portion; blowing into the mouthpiece in a direction generally opposite the forward direction so as to deform the diaphragm inwardly; in response to the diaphragm deformation, releasing the drive element to from the higher state to supply a force to depress a plunger of the pump; in response to the plunger depression, delivering a portion of the medicament into the nasal passages of the user by generating a spray of medicament from the spray port in a spray direction. A dot product of a first vector oriented in the forward direction and a second vector oriented in the spray direction is advantageously a non-zero positive value.

Other aspects of various embodiments of a related inventive device and other related methods are also disclosed in the following description. The various aspects can be used alone or in any combination, as is desired.

### BRIEF DESCRIPTION OF THE DRAWINGS

FIG. 1 shows a cross-sectional view of a nasal drug delivery device according to one embodiment of the present invention in ready configuration.

FIG. 2 shows a cross-sectional view of the device of FIG. 1, with the cocking lever moved to the cocked position.

FIG. 3 shows a more detailed view of one portion of the device of FIG. 2.

FIGS. 4A-B show one embodiment of the cam arm and follower arm, respectively.

FIG. 5 shows a cross-sectional view of the device of FIG. 2 with the diaphragm starting to deform in response to the patient blowing into the mouthpiece.

FIG. 6 shows a cross-sectional view of the device of FIG. 5 releasing medicament in response to the patient blowing into the mouthpiece.

### DETAILED DESCRIPTION OF THE INVENTION

The present invention, in some embodiments, relates to a nasal drug delivery device 10 that includes a pump 50 that is breath actuated to supply drug-containing fluid to a spray port 44 for producing a spray of medicament. The pump 50 is prepared for use by the patient moving a cocking lever 80, and subsequently triggered by the patient blowing into a mouthpiece 130. The blowing action causes a diaphragm 126 to deform, which in turn causes a cam-based firing mechanism 90 to be displaced, resulting in a stored consistent amount of mechanical energy to be applied to the pump's plunger 60. Thus, the pump 50 has a substantially consistent actuation force across dosing events. Additionally, in some embodiments, the forward direction F of the device's housing 20 is oriented with respect to the device's direction of spray S so that the device 10 can be conveniently held by a patient for optimum results.

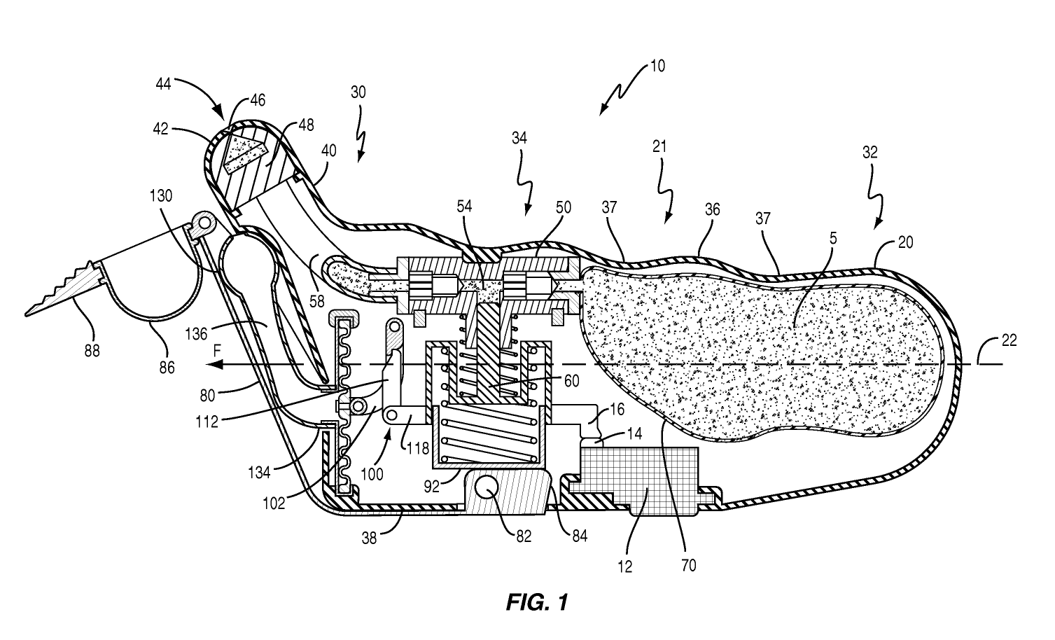

One embodiment of the nasal drug delivery device is shown in FIG. 1, and generally indicated at 10. The device 10 includes a housing 20 having a proximal end portion 30, a distal end portion 32, and an intermediate portion 34. When held in the proper dispensing position by the patient, the proximal end portion 30 is disposed closest to the patient's face and the distal end portion 32 is disposed farthest from the patient's face. Thus, for ease of reference, the direction from the distal end portion 32 to the proximal end portion 30 can be referred to herein as the forward direction F, with the opposite direction referred to as the rearward direction. For the embodiment shown in FIG. 1, the housing 20 is generally elongate along longitudinal axis 22, with an upwardly and forwardly extending protrusion 40 housing spray port 44, as discussed further below. Advantageously, the main portion 21 of the housing 20 is significantly longer along axis 22 than tall, and taller than wide, so as to aid the patient in intuitively positioning the device 10 properly during use. The housing 20 can take a variety of forms, with the upper surface 36 of the housing 20 advantageously including a plurality of finger indentions 37, and a generally flat lower surface 38. The housing forward or proximal portion 30 includes a projection 40 that extends upwardly and forwardly. A spray port 44 is disposed in the tip portion 42 of this projection, and the tip portion 42 is intended to be inserted into the patient's nose during use. As such, the tip portion 42 should be generally rounded and taper appropriately. The forward endface 31 of the housing 20 can include an angled upper portion 310 corresponding to projection 40 and a generally vertical lower section 314. The upper angled portion 310 can advantageously include a suitable recessed area 312 for partially receiving mouthpiece 130, as discussed further below. The housing rear or distal portion 32 can be configured as desired, with a rounded contour believed to be advantageous. The housing 20 can be made of a rigid plastic material and houses most of the elements of device 10. For example, the fluid reservoir 70 can be disposed in the distal portion 32, the pump 50 disposed in the intermediate portion 34, and the spray port 44 disposed in the proximal portion 30.

The reservoir 70 is located in the housing 20 for storage of the liquidous medicament 5. While not required in all embodiments, the reservoir 70 is advantageously formed of a flexible material, such as polyolefin or silicone, so that the reservoir can collapse under atmospheric pressure as the medicament 5 is dispensed. Further, while the reservoir 70 is advantageously permanently disposed fully internal to the housing 20, the reservoir 70 can alternatively be only partially disposed in housing 20, and/or can be removable therefrom, as is desired.

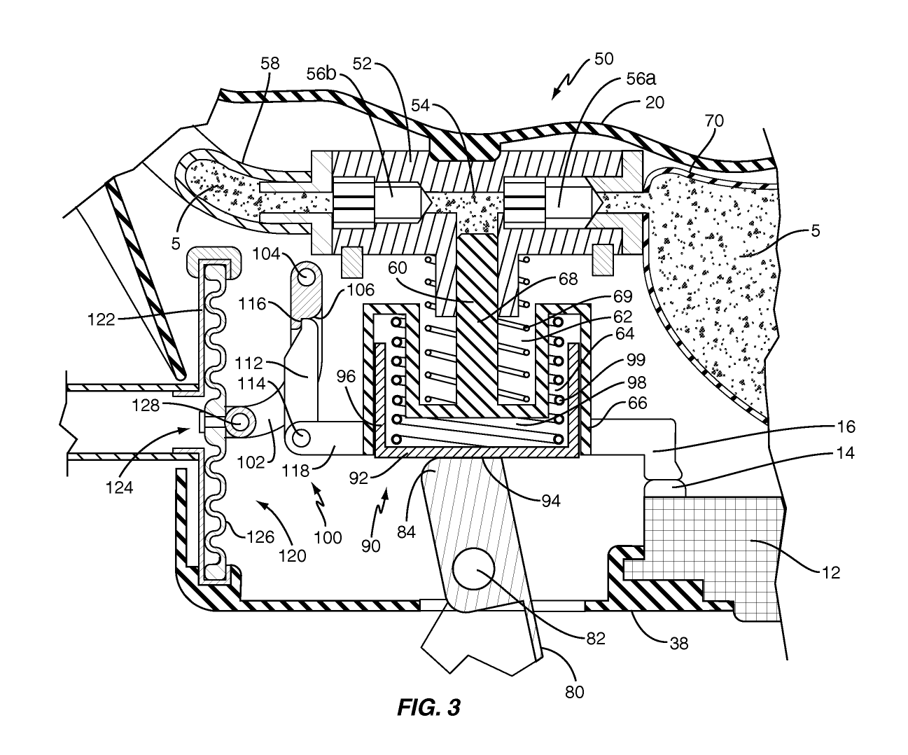

Pump 50 is operatively connected to reservoir 70 and acts to pump medicament from reservoir 70 to spray port 44 when actuated. The pump 50 can be of any type known in the art, but advantageously takes the form of a positive displacement pump such as the elastomeric pump described in U.S. Pat. No. 6,223,746, the disclosure of which is incorporated herein by reference. In one embodiment, the pump 50 includes a main body 52 having a chamber 54, a pair of check valves 56a, 56b, and a plunger 60. See FIG. 3. The check valves 56a, 56b can be elastomeric check valves, ball and spring check valves, reed valves, or other check valves known to those of skill in the art. Inward movement of the plunger 60 toward chamber 54 causes medicament to be forced past check valve 56b and into the delivery channel (e.g., tube) 58 leading to spray port 44. The high pressure nature of this medicament supply causes the medicament 5 to be propelled through the delivery channel 58 and out spray port 44 in spray form. During this process, check valve 56a prevents fluid flow back into the reservoir. Following actuation of the pump 50, plunger 60 is released and begins to move away from the chamber 54, creating a vacuum in chamber 54. The vacuum in chamber 54 causes medicament 5 to flow from reservoir 70 through check valve 56a and into the chamber 54. Medicament 5 is prevented from flowing through check valve 56b because check valve 56b is in the closed position with the vacuum maintaining it as such. Medicament 5 is drawn into the chamber 54 until plunger 60 returns to its rest position. As can be seen, plunger 60 is acted upon by two opposing springs. Reset spring 69 resides between plunger 60 and chamber 54 and acts to urge plunger 60 outward to the ready position. Firing spring 99 resides between cap 92 (discussed below) and plunger 60 and is involved with actuation of plunger 60, as discussed further below. To aid in properly retaining firing spring 99 and reset spring 69, plunger 60 is advantageously formed with a somewhat M-shaped cross-section, with an outer annular channel 64 that faces cap 92 and an inner annular channel 62 that faces pump main body 52. The firing spring 99 rests in outer annular channel 64, while reset spring 69 rests in inner annular channel 62, with the latter generally encircling the central shaft 68 of plunger 60.

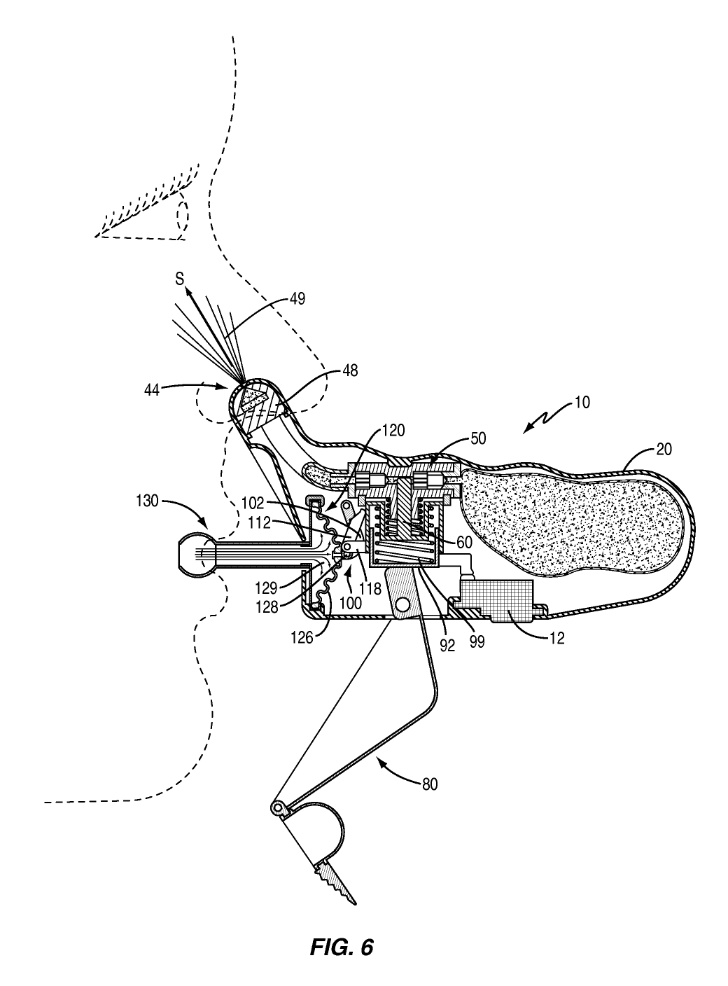

As shown in FIG. 6, medicament 5 is expelled from the device 10 via spray port 44. The spray port 44 includes an opening 46 in housing 20 and a nozzle 48 disposed immediately upstream from opening 46. The opening 46 is disposed on tip 42 of protrusion 40 and is advantageously flared outward, such as by being tapered in a conical fashion. The nozzle 48 is mounted in housing 20 immediately behind the opening 46 and acts to atomize the medicament 5 into a fine mist. The nozzle 48 can take any form known in the art, but advantageously takes the form of a vortex nozzle, such as that described in U.S. Pat. No. 6,418,925, the disclosure of which is incorporated herein by reference. The spray output from nozzle 48 forms a plume 49, advantageously with a vortical flow; for purposes herein, the functional midline of this plume 49 defines a spray direction S.

As can be seen in FIG. 6, this spray direction S for the embodiment of FIG. 1 is upward and outward, away from the main body 21 of housing 20, such that the spray plume 49 follows a path that does not travel back over the housing's main body 21. Thus, the included angle Θ between a vector representing the spray direction S, and a vector oriented in the housing's forward direction F is a non-zero acute angle. Because of this, the dot product of these two vectors is a positive value. This relationship between the main housing body 21 and spray direction S allows the device 10 to be comfortably held in front of the patient's face in an orientation that extends mainly directly away from the patient's face, rather than vertically upward in front of the patient's eye as in some prior art devices. Such ability is believed to encourage greater acceptance by potential patients.

Activation of the pump 50 results in the spraying of medicament 5 from spray port 44. However, because pump 50, in some embodiments, is a positive displacement pump, the force applied to plunger 60 affects the effective fluid pressure of the medicament 5 supplied to spray port 44 which, in turn, affects the particle size distribution of the resulting spray. Some embodiments of the device 10 therefore rely on an indirect actuation of plunger 60 using a compressed firing spring 99, with a consistent amount of energy stored in the compressed spring 99 immediately prior to firing the device, in order to provide a more consistent spray. In some embodiments, the spring 99 is initially compressed a repeatable amount by moving a cocking lever 80, and the spring force is released under the control of a trigger mechanism 100 that is breath actuated in that it is responsive to a patient blowing into a mouthpiece 130. Exemplary embodiments of suitable mechanisms are disclosed below. The disclosed embodiments are believed able to produce effective and repeatable doses of medicament 5 to be applied to the nasal mucosa and turbinates with far superior average particle size when compared with prior art devices. Moreover, the particle size of 20-40 um produced by such embodiments, though small enough to achieve rapid absorption in the nasal turbinates, is not so small that the medicament is readily transported past this region and into the pulmonary system. And, introduction of the medicament 5 to the pulmonary system is further inhibited by the triggering action that requires the patient to blow into the device 10 when the spray is delivered, thereby closing the soft palate in an action similar to that experienced during what is known as the “Valsalva maneuver”.

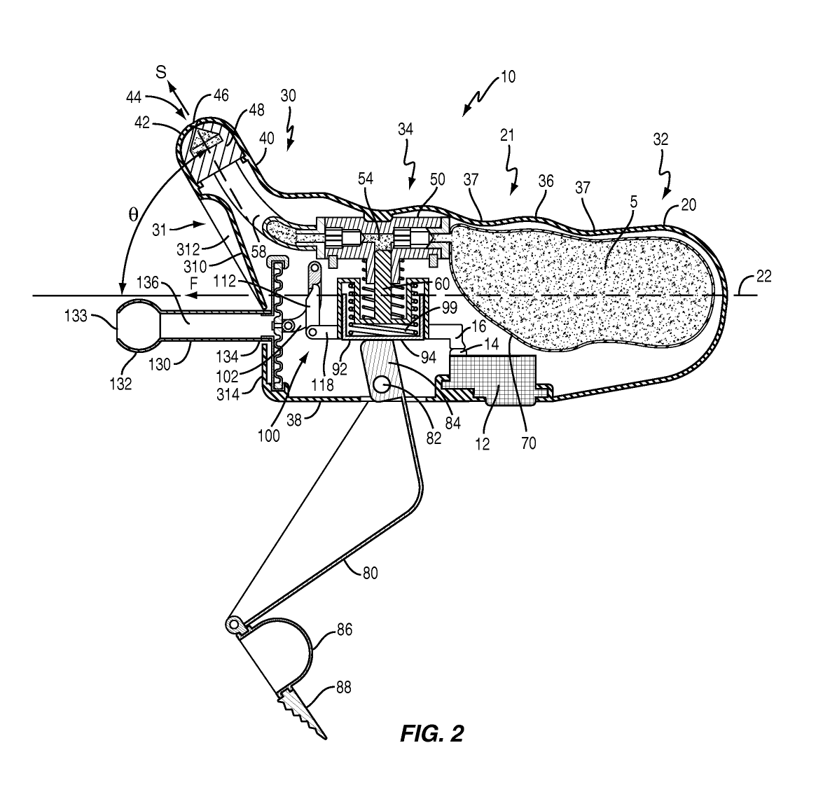

Referring to FIGS. 1-3, an exemplary drive mechanism 90 includes a cocking lever 80, a cap 92, and a firing spring 99. Cocking lever 80 is pivotally mounted to housing 20 so as to be moveable between a storage position (FIG. 1) and a cocked position (FIG. 2). The cocking lever 80 can take any appropriate form, such as an elongate body having a generally C-shaped cross-section that is open toward housing 20, as shown in FIG. 2. Cocking lever 80 pivotally mounts to housing 20 at pivot 82, which is advantageously statically located with respect to housing 20, but can be moveable in some embodiments. The rearward portion of cocking lever 80, proximate pivot 82, includes a cam portion 84 that is eccentric about pivot 82. Cam portion 84 is intended to engage bearing wall 94 of cap 92 to displace cap 92 toward plunger 60 when cocking lever 80 is moved to the cocked position, as discussed further below. In the storage position, the forward portion of cocking lever 80 extends upward in front of housing forward endface 31, as shown in FIG. 1, so as to cover recessed area 312. In the cocked position, the cocking lever 80 extends outward away from housing bottom 38, as shown in FIG. 2.

Cap 92 typically takes the form of a simple cylindrical body closed on one end. The closed end of the cap 92 forms bearing wall 94, and an interior cavity 98 is formed by cap 92, bounded by the bearing wall 94 and associated sidewall 96. Firing spring 99 is disposed in this cavity 98, and abuts against the inner side of bearing wall 94. The other end of firing spring 99 abuts plunger 60. In some embodiments, cap 92 is intended to fit within the plunger, and the radially outer wall 66 of plunger 60 is advantageously sized so as to just receive sidewall 96 of cap 92 with a sliding fit. In other embodiments, cap 92 can fit over plunger 60, and sidewall 96 can include suitable slots for receiving mounting arm 118 and counter arm 16, discussed below.

As indicated above, firing spring 99 abuts against the inner side of bearing wall 94 on one end and plunger 60 on the other end. Advantageously, firing spring 99 is disposed in outer annular channel 64 and cavity 98. The firing spring 99 is elastically deformable. In a compressed state, firing spring 99 stores potential energy. When released, firing spring 99 releases some or all of this potential energy to return to a restored state that has relatively less potential energy. During the firing sequence, the potential energy released by firing spring 99 is used to drive plunger 60 toward pump main body 52 so as to make the pump 50 pump; as such, the firing spring 99 can sometimes be referred to as a drive spring or drive element. In the embodiment illustrated in FIG. 3, firing spring 99 takes the form of a conventional compression coil spring; however, such is not required in all embodiments as discussed further below.

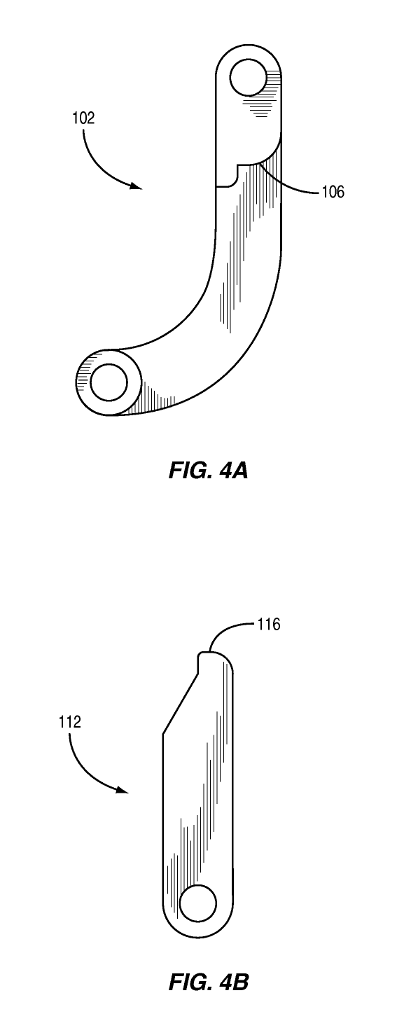

With further regard to FIG. 3, trigger mechanism 100 includes a selectively breakable linkage, a diaphragm assembly 120, and mouthpiece 130. Selectively breakable linkage includes mounting arm 118, follower arm 112, and cam arm 102. Mounting arm 118 is secured to, or integrally formed with, cap 92, so as to move therewith. Follower arm 112 is pivotally mounted to mounting arm 118 at pivot 114 so as to be rotatable thereabout. Follower arm 112 can be advantageously biased toward an upright perpendicular orientation with respect to mounting arm 118 via a suitable biasing means (not shown), such as a small torsion spring. Further, the follower arm 112 and/or mounting arm 118 can advantageously include suitable stop(s) (not shown) for preventing over-rotation of follower arm 112 relative to mounting arm 118. The distal portion of follower arm 112 includes a suitably formed tip 116 for engaging a corresponding lip 106 on cam arm 102. One exemplary configuration for tip 116 is shown in FIG. 4B. Cam arm 102 is pivotally mounted to housing 20 on one end at pivot 104 and pivotally mounted on the other end to diaphragm 126 at pivot 128. Cam arm 102 advantageously has a curved L-shape, with a suitably formed lip 106 for engaging tip 116 of follower arm 112 disposed proximate pivot. One exemplary configuration of cam arm 102 is shown in FIG. 4A. Diaphragm assembly 120 includes a diaphragm frame 122 and a flexible diaphragm 126. Diaphragm frame 122 is mounted to housing 20 and can be formed as an insert or can be integrally formed with housing 20. The diaphragm 126 is elastically deformable and can take any suitable form, such as one formed with a concentric wave profile as shown in FIG. 2. Diaphragm 126 is mounted to diaphragm frame 122 so that a small chamber 129 is formed between the front side of diaphragm 126 and diaphragm frame 122. This chamber 129 is advantageously sealed except for opening 124 which leads to mouthpiece 130. Mouthpiece 130 is generally tubular, with a proximal base 134 and an enlarged distal head 132. A passage 136 extends along the length of mouthpiece 130, with one end open at head aperture 133, and the other end opening to chamber 129. Mouthpiece 130 is advantageously mounted to diaphragm frame 122 via suitable flanges that bound opening 124. Thus, chamber 129 is in fluid communication with head aperture 133 such that air pressure present at head aperture 133 is communicated to the front side of diaphragm 126. Mouthpiece 130 can be formed of a suitably durable, but readily flexible material, such as silicone

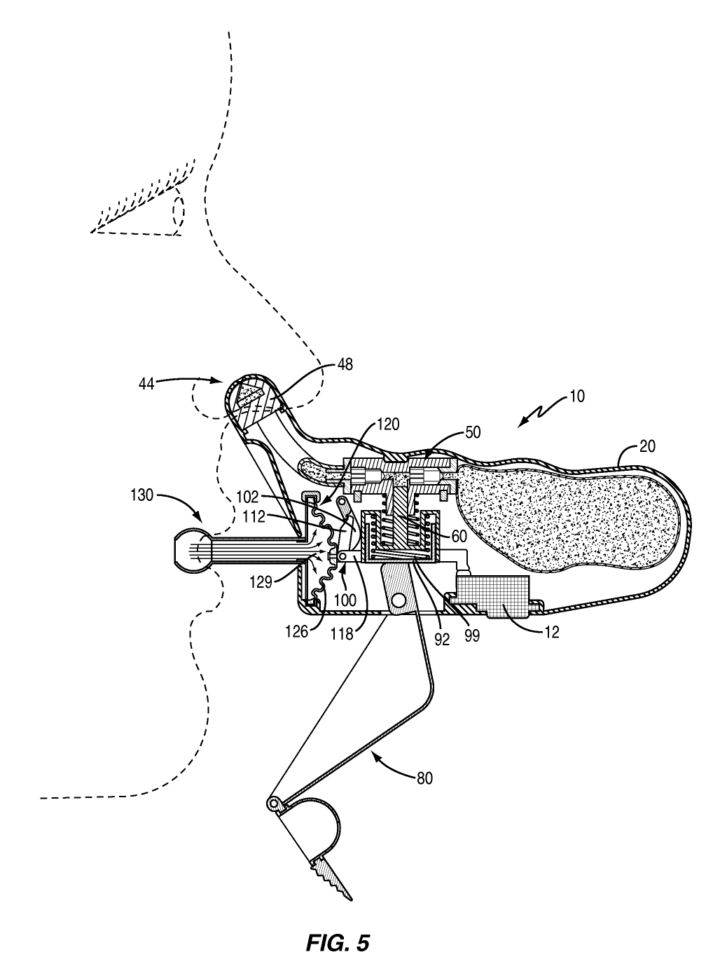

In a storage configuration (FIG. 1), cocking lever 80 is rotated (clockwise in the Figures) so as to overlie forward endface 31, with mouthpiece 130 disposed in recessed area 312 between cocking lever 80 and endface 31. Note that in the storage configuration both reset spring 69 and firing spring 99 are in a less compressed state. The device 10 is prepared for use by rotating cocking lever 80 (counter-clockwise in the Figures) to the cocked position (FIG. 2). With the cocking lever 80 moved out of the way, mouthpiece 130 advantageously automatically springs back to its natural forwardly facing orientation, as shown in FIG. 2. Rotation of cocking lever 80 to the cocked position causes cam portion 84 to press against bearing wall 94, thereby displacing cap 92 towards plunger 60. Plunger 60 is constrained against movement toward pump 50 due to the interaction of follower arm 112 against cam arm 102. More particularly, cam arm 102 is prevented from “vertical” displacement toward pump 50 due to being pinned in location at pivot 104, and follower arm 112 is similarly prevented from displacing due to the locking engagement of tip 116 against lip 106. Because follower arm 112 cannot vertically move, mounting arm 118 likewise cannot move vertically, with the result that plunger 60 is held in its vertical position. Because plunger 60 cannot vertically move, but cap 92 is vertically displaced, firing spring 99 becomes compressed between cap 92 and plunger 60, thereby storing potential energy, and is held against release by trigger mechanism 100. The patient then places the mouthpiece 130 into their mouth and protrusion 40 into one of their nostrils. Holding the device 10 in this position, the patient then blows into mouthpiece 130. The air pressure generated by this blowing is communicated to chamber 129 and presses against the forward face of diaphragm 126. This causes diaphragm 126 to deform inwardly, thereby moving pivot 128 relative to pivot 104. See FIG. 5. This movement causes cam arm 102 to rotate about pivot 104 (counter-clockwise in the Figures), thereby changing the relative angle of tip 116 against lip 106. During this movement, the tip 116 is pressed against the lip 106 by the stored spring force of firing spring 99 urging plunger 60 toward pump 50. As inward movement of diaphragm 126 continues, the cam arm 102 continues rotating until the relative angle between the lip 106 and tip 116 reaches the point where tip 116 eventually becomes unlocked with respect to lip 106, thereby freeing follower arm 112 to rotate (clockwise in the Figures) about pivot 114 to a release position. This frees plunger 60 to move toward pump 50, under the force of firing spring 99. See FIG. 6. The resulting movement of plunger 60 causes pump 50 to pump medicament through tube 58 and out nozzle 48, thereby forming the desired spray plume 49. The patient then stops blowing into mouthpiece 130, which allows diaphragm 126 to return to its natural state, which brings cam arm 102 back to its original orientation. When cocking lever 80 is returned to its storage position, reset spring 69 pushes plunger 60 away from pump main body 52, with the dual effects of loading pump 50 with another charge of medicament 5 from reservoir 70 and moving mounting arm 118 relatively away from pump main body 54. The movement of mounting arm 118 allows follower arm 112 to rotate under its bias back to the perpendicular (locking) position, so that tip 116 can once again engage and lock against lip 106. In addition, firing spring 99 urges cap 92 away from plunger 60 so as to reset the device 10. The cocking and firing regimen can be then be repeated a desired number of times, and the device 10 then stored for later use.

Note that during the actuation process, firing spring 99 is held in a compressed state until the point of release is reached between tip 116 and lip 106, and this compressed state is repeatable across numerous actuations of the device 10. Thus, due to the mechanical arrangement of the firing mechanism 90 and plunger 60, the device 10 is capable of producing a repeatable spray of medicament. Further, the force supplied to the pump 50 from firing spring 99 is not directly proportional to the amount of force with which a patient might press a firing button. Instead, due to the relative positional triggering approach employed, the force applied to the plunger 60 is substantially constant. And, while the firing is triggered by the patient blowing into mouthpiece 130, the substantially constant pump actuation force is supplied to the plunger 60 despite potentially wide variations in how hard the patient is able to blow. Accordingly, both relatively weak and relatively strong patients can be supplied with a medicament spray that is consistent both in amount and in particle size distribution.

Some embodiments of the device 10 can include an optional dose counter 12, and plunger 60 can include a counter arm 16, in order to aid in keeping track of the number of actuations of pump 50. Any form of dose counter known in the art can be used, such as those described in U.S. Pat. Nos. 5,544,647 and 5,622,163, and U.S. patent application Ser. No. 10/625,359, the disclosures of which are incorporated herein by reference. Advantageously, the dose counter 12 is configured so as to be indexed by the sudden movement of counter arm 16 away from a contact 14 connected to the dose counter 12 to increment/decrement dose counter 12 in a conventional fashion. Other functionality can also be incorporated into the dose counter 12 using features known to those of skill in the art.

In some embodiments, a nosepiece cover 86 shaped and configured to cover the end of protrusion 40 can be provided for protecting the spray port 44 during periods of non-use. See FIG. 1. Such a nosepiece cover 86 can advantageously include a suitable side tab 88, if desired, to aid the user in removing and replacing the cover 86. The nosepiece cover 86 can advantageously be pivotally mounted to the free end of cocking lever 80.

The discussion above has assumed that the device 10 includes a coil-type reset spring 69 and a coil-type firing spring 99 for applying their respective biases to plunger 60. However, it should be understood that any form of elastically deformable element known in the art (e.g., compressible foam, leaf spring, etc.) could be used for the desired biasing action, and conventional coil springs are not required in all embodiments. Indeed, while it is believed advantageous to use a conventional compressible element as the firing spring 99, some embodiments of the device 10 can use an extendible element (e.g., extension spring) or a deflectable element (beam-type spring element) alternatively or in addition thereto as the relevant drive element 99. Of course, there can also be multiple drive elements 99.

The discussion above has assumed that the device 10 includes a pivoting cocking lever 80 for loading the drive spring 99 with potential energy; however, such is not required in all embodiments. For example, in some embodiments, there can be no cocking lever 80; instead, the drive spring 99 can be loaded by the patient pressing directly on cap 92, and holding the cap 90 against a suitable stop during the firing sequence. With such an embodiment, the user would likely receive tactile feedback of the firing release of firing spring 99 as the patient blows into mouthpiece 130 to trigger the device. Note that with such an embodiment, the pump action of pump 50, and therefore the resulting spray characteristics, are still not determined by the rate that the cap 90 is pressed. Instead, assuming that the patient has a minimal amount of strength to press/hold cap 92 to/at the desired location, a repeatable amount of pressing force is supplied to plunger 60.

The present invention can be carried out in other specific ways than those herein set forth without departing from the scope and essential characteristics of the invention. Further, the various aspects of the disclosed device and method can be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A nasal drug delivery device comprising:
a housing including a spray port;
a reservoir housing a liquidous medicament;
a selectively actuable pump supported by said housing and operatively connecting said reservoir to said spray port; said pump having a plunger controlling operation thereof;
a deformable drive element operatively connected to said plunger and elastically deformable between a higher potential energy state and lower potential energy state;
a breath actuated triggering mechanism associated with said housing and including a mouthpiece and a diaphragm, said triggering mechanism controlling release of said drive element from said higher potential energy state to said lower potential energy state wherein a pressure differential across said diaphragm, higher towards said mouthpiece, causes said drive element to change from said higher state to said lower state, said plunger moving in response thereto to thereby cause said pump to supply said medicament to said spray port.

2. The device of claim 1 wherein said elastically deformable drive element is relatively compressed in said higher potential energy state and relatively uncompressed in said lower potential energy state.

3. The device of claim 2 wherein said drive element is a spring.

4. The device of claim 3 wherein said drive element is a coil spring.

5. The device of claim 1 further comprising a cocking lever pivotally mounted to said housing for movement between a first position and a second position, wherein movement of said cocking lever to said second position causes said drive element to store potential energy.

6. The device of claim 1 wherein said pump is a positive displacement pump.

7. The device of claim 1 wherein said pump includes a first check valve preventing medicament from traveling from a chamber of the pump to the storage container and a second check valve preventing medicament from traveling from said spray port to the chamber.

8. The device of claim 1 further comprising a dose counter.

9. The device of claim 1 wherein said spray port includes a nozzle that imparts vortical flow to the medicament dispensed from the device.

10. The device of claim 1 further comprising a second elastic element acting on said plunger in opposition to said drive element and providing a reset bias to said plunger.

11. The device of claim 1 wherein said triggering mechanism includes a cam element pivotally attached to said housing and said diaphragm wherein said cam element rotating in response to movement of said diaphragm.

12. The device of claim 11 wherein said triggering mechanism includes a follower element pivotally connected to said plunger for movement therewith, said follower element releasably engaging said cam element.

13. The device of claim 1 wherein said mouthpiece is flexible.

14. The device of claim 1 wherein said medicament in said reservoir is at atmospheric pressure.

15. The device of claim 1 wherein said drive element is a compressible element, wherein said compressible element is relatively compressed in said higher potential energy state and relatively uncompressed in said lower potential energy state, including a second elastic element acting on said plunger in opposition to said compressible element and providing a reset bias to said plunger and a cocking lever pivotally mounted to said housing for movement between a first position and a second position wherein movement of said cocking lever to said second position causes said compressible element to store potential energy and said spray port includes a nozzle imparting vortical flow to the medicament dispensed from the device wherein said triggering mechanism includes a cam element pivotally attached to said housing and said diaphragm, said cam element rotating in response to movement of said diaphragm and a follower element pivotally connected to said plunger for movement therewith, said follower element releasably engaging said cam element wherein said pump is a positive displacement pump; and
wherein said mouthpiece is flexible.

16. A nasal drug delivery device comprising:
a housing having a spray port;
a reservoir housing a liquidous medicament;
a selectively actuable pump supported by said housing and operatively connecting said reservoir to said spray port; said pump having a plunger controlling operation thereof;
an elastically deformable drive element operatively connected to said plunger;
a breath actuated triggering mechanism associated with said housing and comprising a mouthpiece and a diaphragm;
said device moveable between a cocked configuration and a delivery configuration;
wherein said drive element is held in a first relatively higher potential energy state and said plunger is relatively undepressed in said cocked configuration; wherein said drive element is in a relatively lower potential energy state and said plunger is relatively depressed in said delivery configuration wherein blowing into said mouthpiece causes said diaphragm to move so as to release said drive element from said first state and thereby depress said plunger.

17. The device of claim 16 wherein said drive element is elastically compressible.

18. The device of claim 16 further comprising a cocking lever pivotally mounted to said housing for movement between a first position and a second position, wherein movement of said cocking lever to said second position causes said drive element to store potential energy.

19. The device of claim 16 wherein said spray port includes a nozzle imparting vortical flow to the medicament dispensed from the device.

20. The device of claim 16 wherein said triggering mechanism further comprises:
a cam element pivotally attached to said housing and said diaphragm, said cam element rotating in response to movement of said diaphragm; and a follower element pivotally connected to said plunger for movement therewith, said follower element releasably engaging said cam element.

21. A nasal drug delivery device comprising:
a housing having a spray port;
a reservoir housing a liquidous medicament;
a selectively actuable pump supported by said housing and operatively connecting said reservoir to said spray port; said pump having a plunger controlling operation thereof;
a cocking lever pivotally mounted to said housing for movement between a first position and a second position;
a first elastic element disposed operatively between said plunger and said cocking lever, wherein movement of said cocking lever to said second position causes said elastic element to store energy; and
a triggering mechanism comprising a diaphragm and a selectively breakable linkage connecting said diaphragm to said plunger, wherein inward movement of said diaphragm causes said linkage to break, thereby causing said plunger to be depressed by the release of said energy stored in said elastic element.

22. The device of claim 21 wherein said first elastic element is a compression spring.

23. The device of claim 21 further comprising a second elastic element acting on said plunger in opposition to said drive element and providing a reset bias to said plunger.

24. The device of claim 21 wherein said linkage includes a cam element pivotally attached to said housing and said diaphragm; said cam element rotating in response to movement of said diaphragm; and a follower element pivotally connected to said plunger for movement therewith; said follower element releasably engaging said cam element.

25. The device of claim 21 wherein said trigger mechanism includes a mouthpiece in fluid communication with said diaphragm.

26. A method of administering a medicament nasally to a user comprising the steps of:
providing a nasal delivery device, the nasal delivery device comprising:
a housing having a spray port;
a reservoir housing a liquidous medicament;
a selectively actuable pump supported by the housing and operatively connecting the reservoir to the spray port; the pump having a plunger controlling operation thereof;
a cocking lever pivotally mounted to the housing for movement between a first position and a second position;
a first elastic element disposed operatively between the plunger and the cocking lever, storing energy in the first elastic element by moving the cocking lever to the second position while resisting movement of the plunger; and
blowing into a mouthpiece associated with the housing; and, in response thereto, releasing the stored energy to depress the plunger to thereby cause delivery of a portion of the medicament into the nasal passages of a user.

27. The method of claim 26 wherein the delivery of a portion of the medicament into the nasal passages of a user comprises generating a spray having a vortical flow as it exits the spray port.

28. The method of claim 26 wherein the spray port is disposed in a nostril during said blowing.

29. The method of claim 26 wherein the pump comprises a positive displacement pump.

30. The method of claim 26 further comprising actuating a dose counter in response to said releasing of the stored energy.

31. The method of claim 26 wherein said releasing step further comprises the step of deforming a diaphragm in response to said blowing into the mouthpiece.

32. The method of claim 31 wherein said releasing step further comprises the step of breaking a selectively breakable linkage connecting the diaphragm to the plunger in response to inward deformation of the diaphragm.

33. The method of claim 26 wherein the mouthpiece is a flexible mouthpiece extending forwardly from the housing and further comprising flexing the mouthpiece.

34. The method of claim 26 wherein the storing energy in the first elastic element comprises compressing the first elastic element a first amount.

35. The method of claim 26 further comprising the step of thereafter moving the cocking lever to the first position, and, in response thereto, undepressing the plunger.

36. The method of claim 26 further comprising the step of after said moving the cocking lever to the first position, repeating said storing and said blowing steps.

37. A method of administering a medicament nasally to a user comprising the steps of:
providing a nasal delivery device comprising:
a housing comprising a distal end portion and a proximal end portion and comprising a spray port disposed proximate the proximal end portion; the spray port configured to be inserted in a human user's nose; a reservoir housing a liquid medicament; a manually powered pump supported by the housing and operatively connecting the reservoir to the spray port; a deformable drive element operatively connected to the plunger and elastically deformable between a higher potential energy state and lower potential energy state; a breath actuated triggering mechanism associated with the housing and comprising a mouthpiece and a diaphragm; the triggering mechanism controlling release of the drive element from the higher potential energy state to the lower potential energy state;
disposing the proximal end portion proximate the user's face and the distal end portion distal from the user's face; a forward direction defined as extending from the distal end portion toward the proximal end portion;
blowing into the mouthpiece in a direction generally opposite the forward direction so as to deform the diaphragm inwardly;
in response to the diaphragm deformation, releasing the drive element to change from the higher potential state to the lower potential state to supply a force to depress a plunger of the pump;
in response to the plunger depression, delivering a portion of the medicament into the nasal passages of the user by generating a spray of medicament from the spray port in a spray direction.

38. The method of claim 37 wherein a dot product of a first vector oriented in the forward direction and a second vector oriented in the spray direction is a non-zero positive value.

39. The method of claim 37 wherein said generating a spray comprises generating a spray having a vortical flow as it exits the spray port.

40. The method of claim 37 wherein the spray port is disposed in a nostril during said blowing.

41. The method of claim 37 wherein the nasal delivery device further comprises a cocking lever pivotally mounted to the housing for movement between a first position and a second position; the method further comprising, prior to said blowing, storing energy in the drive element in response to movement of the cocking lever to the second position.

42. The method of claim 41 wherein said storing energy in the drive element comprises compressing the drive element a first amount.

43. The method of claim 41 further comprising thereafter moving the cocking lever to the first position, and, in response thereto, undepressing the plunger.

44. The method of claim 43 further comprising, after said moving the cocking lever to the first position, repeating said storing and said blowing steps.
